(19) United States
(12) Patent Application Publication
(10) Pub. No.: US 2012/0053189 A1
(43) Pub. Date: Mar. 1, 2012

(54) BTK INHIBITORS FOR THE TREATMENT OF IMMUNE MEDIATED CONDITIONS

(75) Inventor: David J. Loury, San Jose, CA (US)

(73) Assignee: Pharmacyclics, Inc., Sunnyvale, CA (US)

(21) Appl. No.: 13/171,304

(22) Filed: Jun. 28, 2011

Related U.S. Application Data
(60) Provisional application No. 61/359,281, filed on Jun. 28, 2010.

(51) Int. Cl. A61K 31/519; A61P 29/00; A61P 11/02; A61P 17/00; A61P 7/00; A61P 19/02; A61P 1/00; A61P 37/06; A61P 25/00; A61P 43/00; A61P 7/06; A61P 1/16; A61P 27/02; A61P 9/00; A61P 17/06; A61P 37/08; C07D 487/04

(52) U.S. Cl. 514/262.1; 544/262

(57) ABSTRACT

Disclosed herein are pharmaceutical dosage forms of Brutons tyrosine kinase (Btk) inhibitors. Methods for the preparation of the compounds are disclosed. Methods of using the pharmaceutical dosage forms are disclosed for the treatment of immune mediated conditions and inflammatory diseases or conditions.

### RELATED APPLICATIONS

The present application claims the benefit of priority from U.S. Provisional Patent Application No. 61/359,281 filed Jun. 28, 2010 which is herein incorporated by reference in its entirety.

### FIELD OF THE INVENTION

Described herein are pharmaceutical dosage forms comprising (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one and medicaments containing such compounds, and methods of using such compounds and compositions to treat immune mediated conditions.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

Btk is a key regulator of B-cell development, activation, signaling, and survival (Kurosaki, Curr Op Imm, 2000, 276-281; Schaeffer and Schwartzberg, Curr Op Imm 2000, 282-288). In addition, Btk plays a role in a number of other hematopoetic cell signaling pathways, e.g., Toll like receptor (TLR) and cytokine receptor-mediated TNF-α production in macrophages, IgE receptor (FcepsilonRI) signaling in Mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197:1603-1611; Iwaki et al. (2005), Journal of Biological Chemistry 280(48):40261-40270; Vassilev et al. (1999), Journal of Biological Chemistry 274(3):1646-1656, and Quek et al. (1998), Current Biology 8(20):1137-1140.

### SUMMARY OF THE INVENTION

Described herein is a pharmaceutical dosage form of a Btk inhibitor in therapeutically effective amounts suitable for once a day administration. The daily amount suitable for therapeutic effectiveness in a subject, such as a human, has been shown to be significantly lower than what was previously known. Such low daily amounts provide therapeutic benefit to the subject while limiting the potential side effects typically associated with larger pharmaceutical dosages.

In one aspect is a pharmaceutical dosage form comprising from about 0.1 mg to about 40 mg of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or a pharmaceutically acceptable salt or solvate thereof.

In one embodiment the pharmaceutical dosage form comprises from about 0.1 mg to about 10 mg. In one embodiment the pharmaceutical dosage form comprises from about 0.1 mg to about 5 mg. In one embodiment the pharmaceutical dosage form is suitable for once-a-day administration. In one embodiment the pharmaceutical dosage form is suitable for oral administration. In one embodiment the pharmaceutical dosage form comprises a pharmaceutically acceptable carrier, excipient or binder.

In another aspect is a method for treating an immune-mediated disease or condition comprising administering to a subject in need thereof a therapeutically effective amount of from about 0.1 mg to about 40 mg of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or a pharmaceutically acceptable salt or solvate thereof.

In one embodiment the therapeutically effective amount is from about 0.1 mg to about 10 mg. In another embodiment the therapeutically effective amount is from about 0.1 mg to about 5 mg. In a further embodiment the total amount of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is administered once a day.

In yet a further embodiment the immune-mediated disease or condition is inflammatory bowel conjunctivitis, allergic rhinitis, atopic dermatitis, or idiopathic thrombocytopenic purpura disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia, graft versus host disease, transplantation, transfusion, anaphylaxis, allergy, allergy-related urticaria, type I hypersensitivity, allergic.

In one embodiment the immune-mediated disease or condition is rheumatoid arthritis, lupus, inflammatory bowel disease, allergy, allergy-related urticaria, multiple sclerosis, diabetes, idiopathic thrombocytopenic purpura or transplantation.

In another embodiment the immune-mediated disease or condition is rheumatoid arthritis.

In a further aspect are provided pharmaceutical compositions, which include a therapeutically effective amount of at least one of any of the compounds herein, or a pharmaceutically acceptable salt, pharmaceutically active metabolite, pharmaceutically acceptable prodrug, or pharmaceutically acceptable solvate. In certain embodiments, compositions provided herein further include a pharmaceutically acceptable diluent, excipient and/or binder.

Pharmaceutical compositions formulated for administration by an appropriate route and means containing effective concentrations of one or more of the compounds provided herein, or pharmaceutically effective derivatives thereof, that deliver amounts effective for the treatment, prevention, or amelioration of one or more symptoms of diseases, disorders or conditions that are modulated or otherwise affected by tyrosine kinase activity, or in which tyrosine kinase activity is implicated, are provided. The effective amounts and concentrations are effective for ameliorating any of the symptoms of any of the diseases, disorders or conditions disclosed herein.

In certain embodiments, provided herein is a pharmaceutical composition containing: i) a physiologically acceptable carrier, diluent, and/or excipient; and ii) (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one.

In another aspect, provided herein is the use of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one disclosed herein for inhibiting Bruton's tyrosine kinase (Btk) activity or for the treatment of a disease, disorder, or condition, which would benefit from inhibition of Bruton's tyrosine kinase (Btk) activity.

In some embodiments, compounds provided herein are administered to a human.

In some embodiments, compounds provided herein are orally administered.

In other embodiments, compounds provided herein are used for the formulation of a medicament for the inhibition of tyrosine kinase activity. In some other embodiments, compounds provided herein are used for the formulation of a medicament for the inhibition of Bruton's tyrosine kinase (Btk) activity.

Articles of manufacture including packaging material, a compound or composition or pharmaceutically acceptable derivative thereof provided herein, which is effective for inhibiting the activity of tyrosine kinase(s), such as Btk, within the packaging material, and a label that indicates that the compound or composition, or pharmaceutically acceptable salt, pharmaceutically active metabolite, pharmaceutically acceptable prodrug, or pharmaceutically acceptable solvate thereof, is used for inhibiting the activity of tyrosine kinase(s), such as Btk, are provided.

In certain embodiments, the subject in need is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In further embodiments, the subject in need is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In any of the aforementioned aspects are further embodiments in which administration is enteral, parenteral, or both, and wherein (a) the effective amount of the compound is systemically administered to the mammal; (b) the effective amount of the compound is administered orally to the mammal; (c) the effective amount of the compound is intravenously administered to the mammal; (d) the effective amount of the compound administered by inhalation; (e) the effective amount of the compound is administered by nasal administration; or (f) the effective amount of the compound is administered by injection to the mammal; (g) the effective amount of the compound is administered topically (dermal) to the mammal; (h) the effective amount of the compound is administered by ophthalmic administration; or (i) the effective amount of the compound is administered rectally to the mammal.

In any of the aforementioned aspects are further embodiments comprising single administrations of the effective amount of the compound, including further embodiments in which (i) the compound is administered once; (ii) the compound is administered to the mammal multiple times over the span of one day; (iii) continually; or (iv) continuously.

### BRIEF DESCRIPTION OF THE FIGURES

FIG. 1 presents basophil activation inhibition following anti-IgE stimulation using (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one.

FIG. 2 presents monocyte release inhibition of IL-1β following FcγR stimulation with IgG with (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one and (R)-1-(3-(4-amino-3-(4-phenoxyphenyl)-1H-pyrazolo[3,4-d]pyrimidin-1-yl)piperidin-1-yl)prop-2-en-1-one.

FIG. 3 presents non-inhibition of IgG opsonized Zymosan particle phagocytosis by murine macrophage RAW cells at the concentrations tested, up to 10,000 nM using (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one and (R)-1-(3-(4-amino-3-(4-phenoxyphenyl)-1H-pyrazolo[3,4-d]pyrimidin-1-yl)piperidin-1-yl)prop-2-en-1-one.

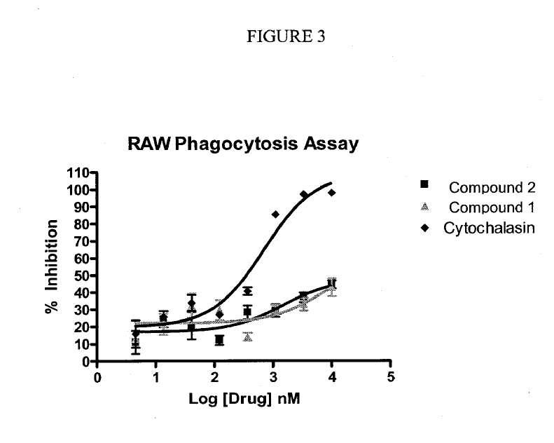

FIG. 4 presents reversal of arthritic clinical scores in a murine CIA model using (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one.

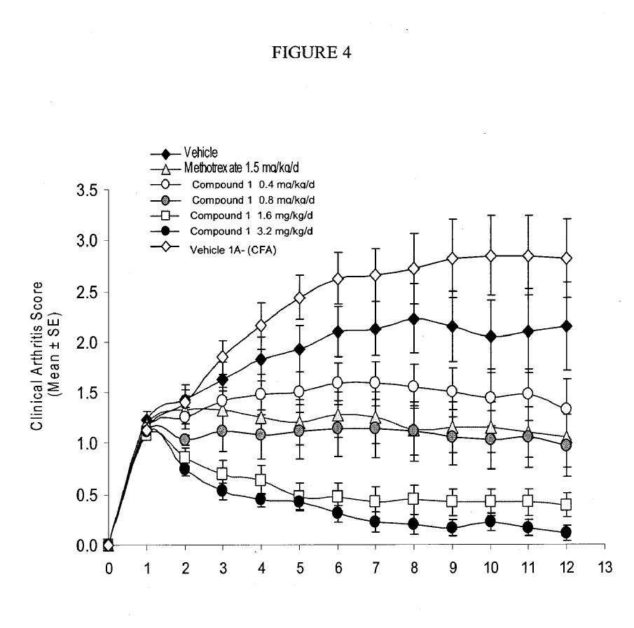

FIG. 5 shows complete suppression of arthritic inflammation in a Collagen-induced Arthritis Model (CAIA) in DBA/1 mice.

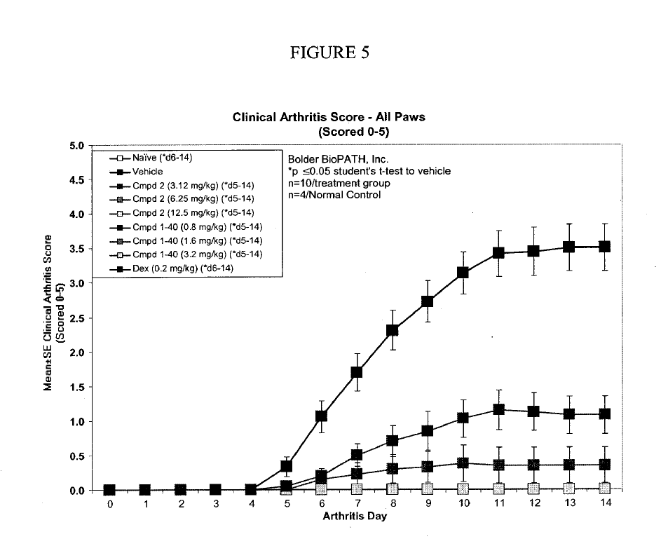

FIG. 6 shows hepatic extraction ratio of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one in rats (0.289.)

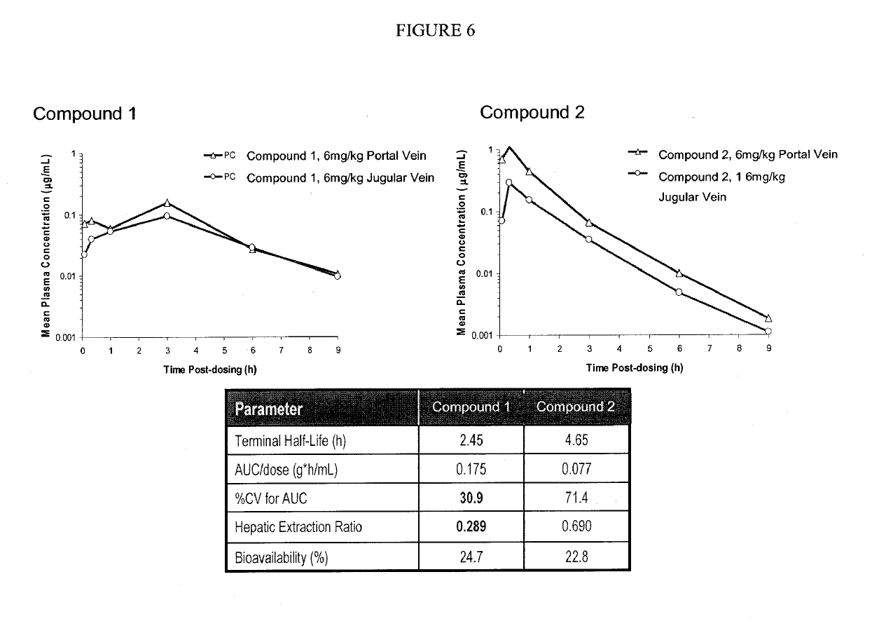

FIG. 7 shows allometric scaling of CL/F vs. Body Weight in multiple species is linear for (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one.

FIG. 8 shows non-inhibition of T cell activation measured by CD69 expression following stimulation with anti-CD3/28 at concentrations up to 2000 nM in washout experiments using (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one (Compound 1) and (R)-1-(3-(4-amino-3-(4-phenoxyphenyl)-1H-pyrazolo[3,4-d]pyrimidin-1-yl)piperidin-1-yl)prop-2-en-1-one (Compound 2).

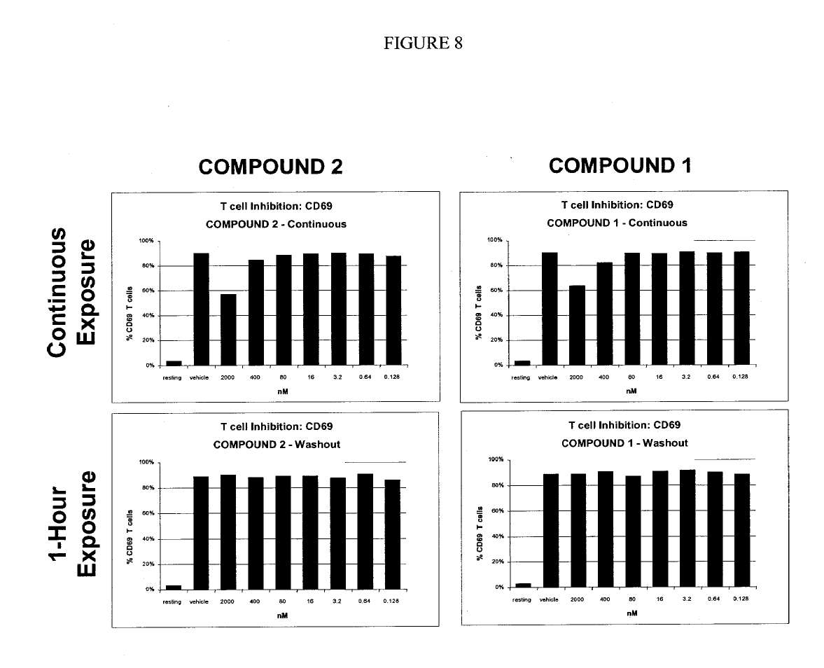

### INCORPORATION BY REFERENCE

All publications and patent applications mentioned in this specification are herein incorporated by reference to the same extent as if each individual publication or patent application was specifically and individually indicated to be incorporated by reference for the purposes cited.

### DETAILED DESCRIPTION OF THE INVENTION

Rheumatoid arthritis (RA) is a chronic, systemic disorder classified as an autoimmune disease. Symptoms of RA manifest as inflammation in the joints (arthritis) and can quickly progress to involve painful swelling and deterioration of the surrounding tissues and organs. Thus, RA can be very debilitating as patients may experience substantial loss of function and mobility in the hands, wrists, and feet. Though there is no known cure for RA, there are different types of treatments available for to alleviate symptoms and modifying the disease progression. Unfortunately, many of the available therapies have significant side effects and long-term risks.

Autoimmune diseases such as RA are characterized with pathogenic autoantibody production (such as the presence of Rheumatoid Factor and antibodies to citrullinated peptides) as well as immune-complex mediated activation of Fc-gamma signaling pathways resulting in pro-inflammatory cytokine production of effector cells (macrophages, neutrophils, mast cells) leading to tissue destruction primarily in the joints. Btk is key component of BCR signaling in B cells and Fc-gamma signaling in myeloid an origin in the bone marrow or spinal cord, or a resemblance to the marrow or spinal cordcells. Inhibition of Btk activity is expected to reduce two major components of autoimmune diseases: the pathogenic autoantibody production by B cells and the pro-inflammatory cytokines produced by myeloid cells.

The methods described herein include administering to a subject in need a composition containing a therapeutically effective amount of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one. Without being bound by theory, the diverse roles played by Btk signaling in various hematopoietic cell functions, e.g., B-cell receptor activation, suggests that small molecule Btk inhibitors such as (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one are useful for reducing the risk of or treating a variety of diseases affected by or affecting many cell types of the hematopoetic lineage including, e.g., autoimmune diseases, heteroimmune conditions or diseases, inflammatory diseases, (e.g., B-cell proliferative disorders), and thromboembolic disorders.

In some embodiments, the methods described herein are used to treat an autoimmune disease, which includes, but is not limited to, rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, and vulvodynia.

In some embodiments, the methods described herein are used to treat heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In further embodiments, the methods described herein are used to treat an inflammatory disease, which includes, but is not limited to asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In further embodiments, the methods described herein are used to treat thromboembolic disorders, which include, but are not limited to myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses.

In another embodiment, the methods described herein are used to treat inflammatory synovitis, pannus formation, syunovial fluid cytokines, cartilage damage and bone erosion.

Symptoms, diagnostic tests, and prognostic tests for each of the above-mentioned conditions are known in the art. See, e.g., Harrison's Principles of Internal Medicine®,” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

A number of animal models of are useful for establishing a range of therapeutically effective doses of irreversible Btk inhibitor compounds for treating any of the foregoing diseases.

For example, dosing of the compositions described herein for treating an autoimmune disease can be assessed in a mouse model of rheumatoid arthritis. In this model, arthritis is induced in Balb/c mice by administering anti-collagen antibodies and lipopolysaccharide. See Nandakumar et al. (2003), Am. J. Pathol 163:1827-1837.

Animal models for treatment of thromboembolic disorders are also known.

The therapeutic efficacy of the compound for one of the foregoing diseases, in some embodiments, is optimized during a course of treatment. For example, in one embodiment, a subject being treated undergoes a diagnostic evaluation to correlate the relief of disease symptoms or pathologies to inhibition of in vivo Btk activity achieved by administering a given dose of a composition described herein. In other embodiments, cellular assays are used to determine in vivo activity of Btk in the presence or absence of a composition described herein. For example, since activated Btk is phosphorylated at tyrosine 223 (Y223) and tyrosine 551 (Y551), phospho-specific immunocytochemical staining of P-Y223 or P-Y551-positive cells, in some embodiments, is used to detect or quantify activation of Bkt in a population of cells (e.g., by FACS analysis of stained vs unstained cells). See, e.g., Nisitani et al. (1999), Proc. Natl. Acad. Sci, USA 96:2221-2226.

### Compounds

The compounds described herein, such as compound (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one having the structure:

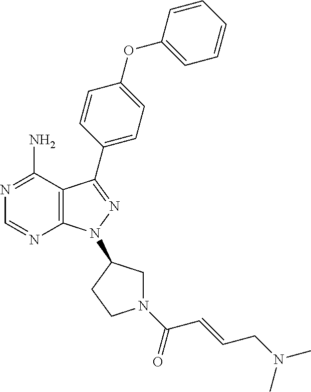

and methods of preparing them are described in U.S. Pat. No. 7,514,444 and is incorporated by reference in its entirety.

### Pharmaceutical Dosage Forms

Described herein are orally-administered pharmaceutical dosage forms of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or a pharmaceutically acceptable salt or solvate thereof suitable for daily administration. In one embodiment, the dosages employed for treatment using (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is from about 0.01 mg per day to about 50 mg per day. In one embodiment, the daily dosage is from about 0.1 mg per day to about 30 mg per day. In another embodiment, the daily dosage is from about 0.1 mg per day to about 20 mg per day. In a further embodiment, the daily dosage is from about 0.1 mg per day, about 0.2 mg per day, about 0.3 mg per day, about 0.4 mg per day, about 0.5 mg per day, about 0.6 mg per day, about 0.7 mg per day, about 0.8 mg per day, about 0.9 mg per day and about 1.0 mg per day. In another embodiment, the daily dose of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or a pharmaceutically acceptable salt or solvate is from about 1.1 mg per day, about 1.2 mg per day, about 1.3 mg per day, about 1.4 mg per day, about 1.5 mg per day, about 1.6 mg per day, about 1.7 mg per day, about 1.8 mg per day, about 1.9 mg per day, and about 2.0 mg per day. In yet another embodiment is a pharmaceutical dosage form wherein the daily dose of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is about 0.1 mg per day to about 1.0 mg per day, about 1.0 mg per day, to about 2.0 mg per day, about 1.0 mg per day to about 3.0 mg per day, about 1.0 mg per day to about 4.0 mg per day, about 1.0 mg per day to about 5.0 mg per day. In another embodiment, the daily dose is less than or equal to 10 mg per day.

In yet another embodiment, the daily dosage is from about 5 mg per day to about 10 mg per day. In another, about 5 mg per day to about 15 mg per day. In yet another, about 10 mg per day to about 20 mg per day.

In some embodiments, (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one used for the methods described herein inhibits Btk or a Btk homolog kinase activity with an in vitro IC₅₀ of less than 10 μM. (e.g., less than 1 μM, less than 0.5 μM, less than 0.4 μM, less than 0.3 μM, less than 0.1, less than 0.08 μM, less than 0.06 μM, less than 0.05 μM, less than 0.04 μM, less than 0.03 μM, less than less than 0.02 μM, less than 0.01, less than 0.008 μM, less than 0.006 μM, less than 0.005 μM, less than 0.004 μM, less than 0.003 μM, less than less than 0.002 μM, less than 0.001, less than 0.00099 μM, less than 0.00098 μM, less than 0.00097 μM, less than 0.00096 μM, less than 0.00095 μM, less than 0.00094 μM, less than 0.00093 μM, less than 0.00092, or less than 0.00090 μM).

### Further Forms of Compounds

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed) by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

It should be understood that a reference to a salt includes the solvent addition forms or crystal forms thereof, particularly solvates or polymorphs. Solvates contain either stoichiometric or non-stoichiometric amounts of a solvent, and are often formed during the process of crystallization with pharmaceutically acceptable solvents such as water, ethanol, and the like. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for this disclosure.

A pharmaceutical composition, as used herein, refers to a mixture of a compound described herein, such as, for example, (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one), with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

### Examples of Methods of Dosing and Treatment Regimens

In some embodiments, the pharmaceutical dosage form is administered to a subject in need once a day. In another embodiment, the dosage form is suitable for once a week administration.

The compounds described herein can be used in the preparation of medicaments for the inhibition of Btk or a homolog thereof, or for the treatment of diseases or conditions that would benefit, at least in part, from inhibition of Btk or a homolog thereof. In addition, a method for treating any of the diseases or conditions described herein in a subject in need of such treatment, involves administration of pharmaceutical compositions containing at least one compound of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one, described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Treatments

In certain instances, it may be appropriate to administer at least pharmaceutical composition described herein in combination with another therapeutic agent. By way of example only, if one of the side effects experienced by a patient upon receiving one of the compounds described herein is nausea, then it may be appropriate to administer an anti-nausea agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

For combination therapies described herein, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In addition, the pharmaceutical dosages described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of Btk, or in which Btk is a mediator or contributor to the symptoms or cause.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically may include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent.

Overview:

In Btk biochemical assays, (R)-1-(3-(4-amino-3-(4-phenoxyphenyl)-1H-pyrazolo[3,4-d]pyrimidin-1-yl)piperidin-1-yl)prop-2-en-1-one and (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one have an IC₅₀ of 0.5 nM and 1-2 nM, respectively. (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one was a more selective inhibitor of Btk than either (R)-1-(3-(4-amino-3-(4-phenoxyphenyl)-1H-pyrazolo[3,4-d]pyrimidin-1-yl)piperidin-1-yl)prop-2-en-1-one or dasatinib, with no significant inhibition of VEGFR2, EGFR, JAK(1, 2 or 3), or Abl. Both (R)-1-(3-(4-amino-3-(4-phenoxyphenyl)-1H-pyrazolo[3,4-d]pyrimidin-1-yl)piperidin-1-yl)prop-2-en-1-one and (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one were potent inhibitors of human B cell activation following BCR stimulation by anti-IgM with an EC₅₀ of 2 nM while failing to inhibit T cell activation at concentrations up to 2,000 nM. Both compounds also inhibited anti-IgE mediated upregulation of CD63 in human whole blood basophils with an EC₅₀ of 20-100 nM. In addition, (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one inhibited cytokine release from human monocytes at 20-100 nM but did not inhibit IgG-mediated phagocytosis at concentrations up to 10,000 nM. In vivo, both Btk inhibitors dose-dependently inhibited inflammatory synovitis, pannus formation, synovial fluid cytokines, cartilage damage and bone erosion in both preventive and established murine collagen-induced arthritis (CIA) models. (R)-1-(3-(4-amino-3-(4-phenoxyphenyl)-1H-pyrazolo[3,4-d]pyrimidin-1-yl)piperidin-1-yl)prop-2-en-1-one and (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one inhibited overt manifestations of arthritis in mice with EC₅₀ values of 2.23 and 0.61 mg/kg/day, respectively. In a murine collagen-antibody-induced arthritis model (CAIA model), (R)-1-(3-(4-amino-3-(4-phenoxyphenyl)-1H-pyrazolo[3,4-d]pyrimidin-1-yl)piperidin-1-yl)prop-2-en-1-one and (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one completely suppressed the development of arthritis at doses of 6.25 and 0.8 mg/kg/day, respectively. In glutathione binding assays, the rate of glutathione conjugation was 20 fold lower for (E)-1-((R)-3-(4-amino-3-(4-phenoxyphenyl)-1H-pyrazolo[3,4-d]pyrimidin-1 (dimethylamino)but-2-en-1-one than for (R)-1-(3-(4-amino-3-(4-phenoxyphenyl)-1H-pyrazolo[3,4-d]pyrimidin-1-yl)piperidin-1-yl)prop-2-en-1-one. In human liver microsomes, the half-life of Compound 2 was 2.5 mM compared to 19.2 min for (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one. Pharmacokinetic studies in rats showed that the bioavailability of (R)-1-(3-(4-amino-3-(4-phenoxyphenyl)-1H-pyrazolo[3,4-d]pyrimidin-1-yl)piperidin-1-yl)prop-2-en-1-one and (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one was 22.8% and 24.7%, respectively. Hepatic extraction ratio, a measurement of first-pass metabolism, was 0.690 and 0.289 for (R)-1-(3-(4-amino-3-(4-phenoxyphenyl)-1H-pyrazolo[3,4-d]pyrimidin-1-yl)piperidin-1-yl)prop-2-en-1-one and (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one, respectively. Based on pharmacokinetic/efficacy relationships in mice and interspecies scaling of clearance, the daily efficacious dosage of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one was estimated to be ≦10 mg/patient/day.

Examples 1-8 explain more fully the results described in the overview above.

### Example 1

### Basophil Assays

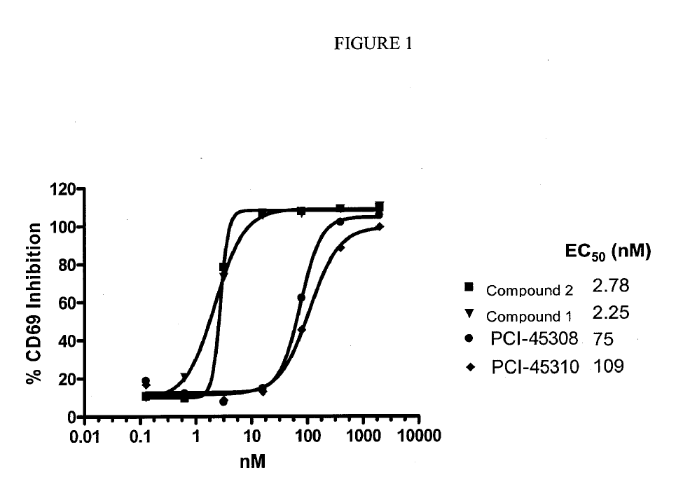

Fresh whole blood was collected on the day of the experiment from a healthy volunteer. Whole blood is stimulated with 10 ng/mL of rhIL-3 (Invitrogen) and 10 μM of anti-IgE (Beckman Coulter) in Basophil Stimulation Buffer (20 mM HEPES pH 7.4, 125 mM NaCl, 5 mM KCl, 2.5 mM CaCl₂, 1 mM MgCl₂, 0.5% Glucose, 0.1% BSA) at 37° C. for 15 min. The reaction was terminated by the addition of EDTA. The stimulated whole blood was then stained with a fluorescent three-antibody cocktail (FITC-Anti-CD63/PerCP-Anti-HLA-DR/PE-Anti-CD123) (BD Biosciences) at room temperature in the dark for 15 min. Following staining, blood was treated with 1×FACS Lysing solution (BD Biosciences) for 15 minutes to lyse the RBC. The lysed RBC supernatant was removed following centrifugation for 5 mM. The cell pellet was resuspended and fixed in 0.5% paraformaldehyde in PBS. The percentage of basophil degranulation was determined by the % of CD63 cells that were CD123 positive and HLA-DR negative. (E)-1-4R)-3-(4-amino-3-(4-phenoxyphenyl)-1H-pyrazolo[3,4-d]pyrimidin-1-yl)pyrrolidin-1-yl)-4-(dimethylamino)but-2-en-1-one inhibited CD63 expression following anti-IgE stimulation in whole blood basophil assays (FIG. 1) with EC₅₀ of about 92 nM.

### Example 2

### Human Monocyte Cytokine Release Assays

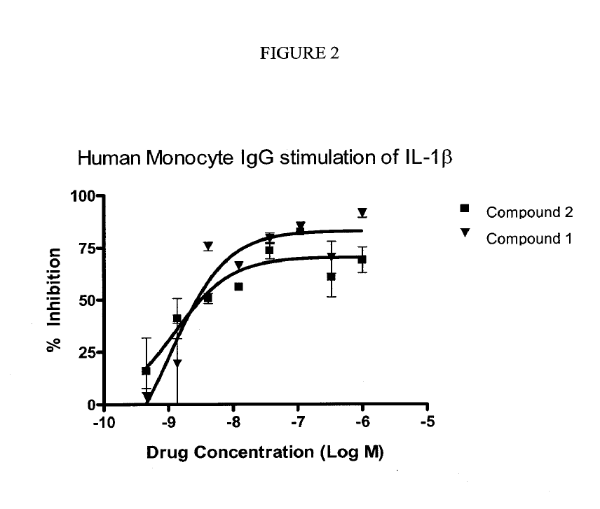

Human monocytes were isolated from Ficoll-gradient purified PBMC from healthy human volunteers using negative selection with EasySep (StemCells) magnetic beads. CD14+monocytes were then stimulated with IFN-gamma for 6 days, and then 2×10⁶ cells were plated onto 10 μg/ml of human IgG (R&D Biosystems) coated onto Immulon 4 ELISA plates. Serial dilutions of X or (R)-1-(3-(4-amino-3-(4-phenoxyphenyl)-1H-pyrazolo[3,4-d]pyrimidin-1-yl)piperidin-1-yl)prop-2-en-1-one were added to the cells and incubated at 37 C for 24 hrs. Supernatants were collected from the plates and cytokines levels were determined with ELISA kits. Both (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or (R)-1-(3-(4-amino-3-(4-phenoxyphenyl)-1H-pyrazolo[3,4-d]pyrimidin-1-yl)piperidin-1-yl)prop-2-en-1-one suppressed the release of IL-1b from human monocytes following IgG stimulation (FIG. 2).

### Example 3

### IgG Mediated Phagocytosis of Zymosan Particles

Prelabeled Zymosan particles were first opsonized with mouse IgG at 37 C for 30 min, and then centrifuged down and washed several times with PBS. The Zymosan particles are the resuspended in PBS. RAW264.7 cells are treated with serial dilutions of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or Cytochalasin (an actin polymerization inhibitor and positive control for inhibition of phagocytosis. Opsonized Zymosan is then added to the RAW cells, mixed and incubated at 37 C for 2 hrs. The culture medium and excess particles are removed by multiple steps of washing. The internalized particles are quantitated following fixation with paraformaldehyde and permeabilization with Triton-100.

### Example 4

### Inhibition of Clinical Arthritic Scores in a Murine Collagen-induced Arthritis Model Using (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one

Male mice 7 weeks of age were purchased from Harlan Laboratories, and acclimated for a minimum of 3 days. Mice were immunized on Day 0 with 150 μg of bovine collagen II (Chondrex Inc) in CFA (Sigma), and boosted with 100 μg of bovine collagen II in IFA on day 21. Fore and hind paws of mice were scored daily with a clinical scoring system using the following criteria:

0=normal 1=1 hind or fore paw joint affected
with minimal diffuse swelling and erythema (redness of the skin due to congestion of the capillaries)

2=2 hind or fore paw joints affected
with minimal diffuse swelling and erythema (redness of the skin due to congestion of the capillaries)

3=3 hind or fore paw joints affected
with minimal diffuse swelling and erythema (redness of the skin due to congestion of the capillaries)

4=minimum of 4 joints affected
marked diffuse swelling and erythema (redness of the skin due to congestion of the capillaries)

5=minimum entire paw,
unable to flex (Severe diffuse swelling and erythema redness of the skin due to congestion of the capillaries

Mice were enrolled randomly into treatment groups when a minimum mean paw score of 1.0 was established. The treatments started on the day of enrollment and continued for 12 days. Clinical scores were monitored daily for each animal in the treatment groups. (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one was effective in ameliorating clinical arthritic symptoms with an EC₅₀ of 0.61 mg/kg/day. The two higher doses of 1.6 and 3.2 mg/kg/day (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one treatments reversed clinical arthritic symptoms in the CIA model, and the reversal of clinical arthritis was observed 1 day following drug administration. In contrast, methotrexate treatments of 1.5 mg/kg/day stabilized disease without further progression of arthritic swelling for the duration of the treatment.

### Example 5

### Complete Suppression of Arthritic Inflammation in a Collagen-Induced Arthritis Model (CAIA Model)

DBA/1 mice were injected intravenously with 2 mg of Arthrogen-CIA 5 mouse anti-bovine collagen (Chondrex) on day 0. Three days later, these mice were injected IP with 50 μg of LPS. Mice were randomized on day 0, and placed into one of the treatment groups and treatments initiated on day 0 until the end of the study (day 14): vehicle, (R)-1-(3-(4-amino-3-(4-phenoxyphenyl)-1H-pyrazolo[3,4-d]pyrimidin-1-yl)piperidin-1-yl)prop-2-en-1-one (3.125, 6.25 or 12.5 mg/kg), (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one (0.8, 1.6 or 3.2 mg/kg).

Mice were monitored for clinical symptoms daily with the criteria below.

### Clinical Scoring Criteria for Fore and Hind Paws

0=normal
1=1 hind or fore paw joint affected
2=2 hind or fore paw joints affected
3=3 hind or fore paw joints affected
4=moderate (erythema and moderate swelling, or =4 digit joints affected)
5=severe (diffuse erythema and severe swelling entire paw, unable to flex digits)

These studies suggested (E)-1-((R)-3-(4-amino-3-(4-phenoxyphenyl)-1H-pyrazolo[3,4-d]pyrimidin-1-yl)pyrrolidin-1-yl)-4-(dim ethylamino)but-2-en-1-one treatments (0.8, 1.6 and 3.2 mg/kg/day) completely prevented and suppressed clinical arthritis in this model while 0.2 mg/kg of dexamethasone was able to partially suppress clinical scores and maintained an average clinical scores of 1 on day 14. The average clinical scores in the vehicle group were 3/5 at the end of the study.

### Example 6

### Reduced Hepatic Extraction Ratio of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one Compared to (R)-1-(3-(4-amino-3-(4-phenoxyphenyl)-1H-pyrazolo[3,4-d]pyrimidin-1-yl)piperidin-1-yl)prop-2-en-1-one.

Male CDIGS rats with jugular or portal vein cannula were orally administered with (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one) and plasma were collected and analyzed with standard LC/MS methods. Plasma concentration data were evaluated using the computer program WinNonlin (Professional Edition, Pharsight Corporation, version 5.01). The analyses were performed using nominal sample times and a noncompartmental method with uniform weighting. Pharmacokinetic parameter estimates included terminal half-life and area under the concentration-time curve (AUC). Extraction ratios were calculated as 1-(AUC systemic÷AUC portal).

### Example 7

### Estimation of Efficacious Daily Dosage of Compound (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one

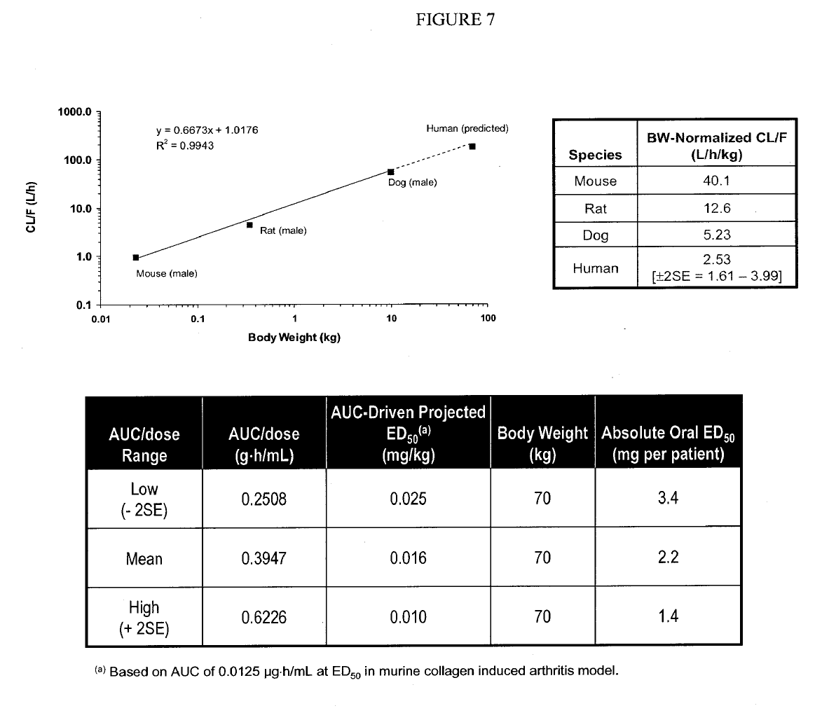

There is an excellent scaling of clearance (dose/AUC) within mice, rats and dogs, and therefore exposure to dose projection in humans is likely precise. Based on interspecies scaling of clearance, the human BW normalized CL/F was determined to be about 2.53 (FIG. 7). Using the ED₅₀ established in mouse CIA models, the putative projected human ED₅₀ will be 0.016 mg/kg which translates into a daily 2.2 mg dose of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one.

### Example 8

### B and T cell Assays

CD20+ B and CD3+ T cells were purified by negative selection (RosetteSep, >90% purity) from Buffy coat PBMC and viably frozen in 10% DMSO. Cells were thawed at 37° C. and maintained in growth media (RPMI media containing 10% FCS). B cells were stimulated with goat anti-human IgM F(ab′)2 (10 ug/ml, Invitrogen) and T cells were stimulated with anti-CD3/CD28 coated beads (Dynabeads) at a 1:1 bead/cell ratio. Cells were stained with PE-CD69 (BD) and analyzed by flow cytometry, gating on viable lymphocytes. (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or (R)-1-(3-(4-amino-3-(4-phenoxyphenyl)-1H-pyrazolo[3,4-d]pyrimidin-1-yl)piperidin-1-yl)prop-2-en-1-one at concentrations below 10 μM did not decrease B or T cell viability during the course of the experiment. For washout experiments, cells were rinsed 3× in 10 volumes of growth media, a protocol that was confirmed to completely wash away inhibition of BCR signaling by a reversible Btk inhibitor.

### Example 9

### Clinical Trial Assessing the Safety and Efficacy of a Pharmaceutical Dosage Form of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one in Adult Patients with Established Rheumatoid Arthritis

Purpose: This study will assess the safety, efficacy, and response to treatment using the American College of Rheumatology criteria of 20% improvement in symptoms (ACR20) in adult patients with established rheumatoid arthritis using (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one

Study Type: Interventional

Study Design:
Allocation: Randomized
Control: Placebo Control
Endpoint Classification: Safety/Efficacy Study
Intervention Model: Parallel Assignment
Masking: Double Blind (Subject, Caregiver, Investigator, Outcomes Assessor)
Primary Purpose: Treatment

Primary Outcome Measures:
Response to treatment (ACR20) in adult patients with established rheumatoid arthritis(RA) [Time Frame: at 6 weeks]

Eligibility: Ages eligible for study: 18 years to 75 years; Genders eligible for study: Both;

Inclusion Criteria:

RA patients:
Male and female patients aged 18-75 years (inclusive);
Body weight between 50 and 100 kg (inclusive);
Post menopausal or surgically sterile female patients are allowed. Female patients of child-bearing potential may participate if they are already on a stable dose of methotrexate. Additional birth control details to be provided at screening. Male patients must use an effective contraception method during the study and at least for 2 months following the completion/discontinuation of the study;
Diagnosis of RA, classified by American Rheumatism Association 1987 revised criteria. Disease duration of at least 6 months is essential;
Functional status class I, II or III classified according to the American College of Rheumatology 1991 revised criteria;
Active disease evaluation (≧6 tender and ≧6 swollen joints);
Prior treatment with 1-3 disease-modifying anti-rheumatic drugs (DMARDs)—Patients should have failed at least 1 DMARD but should not be deemed “refractory to all therapies”. It is expected that patients are on a current treatment with methotrexate≦25 mg/week and with the current dose stable for at least 3 months, however patients who did not tolerate MTX may also be considered. All patients will take folic acid 1 mg daily, or 5 mg weekly post MTX dose, to minimize toxicity, according to local guidelines. In addition to methotrexate, patients may be on either a stable dose of non-steroidal anti-inflammatory drugs (NSAIDs) and/or a stable dose of oral corticosteroids (prednisone or equivalent≦10 mg daily) for at least 4 weeks prior to randomization. Patients who failed any DMARDs will be allowed;
Negative purified protein derivative (PPD) tuberculin skin test reaction (PPD 5 tuberculin units or as according to local standard practice);

Exclusion Criteria:

RA patients:
Previous treatment with anti-TNF-α or anti IL-1 therapy (or other biological therapy), immunosuppressive agents such as cyclosporine, mycophenolate or tacrolimus. The following washout period will be required for such patients to be eligible to participate in the trial.
   1. 2 months washout prior to screening for etanercept or adalimumab;
   2. 3 months washout prior to screening for infliximab;
   3. 3 months washout prior to screening for rituximab;
   4. 1 month washout prior to screening for cyclosporine, mycophenolate and tacrolimus;
If patient has been discontinued from other DMARDs (disease modifying antirheumatic drugs) for lack of efficacy or toxicity, the patient should be at least 1 month off the agent;
Patients with congestive heart failure, QT prolongation syndrome or poorly controlled diabetes mellitus. Patients with a history of QTc prolongation will be excluded;
Patients who have received intra-articular or systemic corticosteroid injections having been required for treatment of acute RA flare (not being part of a regular therapeutic regimen) within 4 weeks prior to randomization;
Exclusion criteria 2-6 of the Health Volunteer section also applies her

### Example 10

### Clinical Trial of a Pharmaceutical Dosage Form of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one in Subjects with Moderately to Severely Active Systemic Lupus Erythematosus (SLE)

Purpose The purpose of this study is to evaluate the safety and tolerability of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one in adult subjects with moderately to severely active systemic lupus erythematosus (SLE).

Study Type: Interventional

Study Design:
Allocation: Randomized
Control: Active Control
Endpoint Classification: Safety Study
Intervention Model: Parallel Assignment
Masking: Double Blind (Subject, Investigator)
Primary Purpose: Treatment

Primary Outcome Measures:
The safety and tolerability of a pharmaceutical dosage form of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is assessed primarily by summarizing treatment-emergent adverse events (AEs) and serious adverse events (SAEs) [Time Frame: Study Day 169]

Secondary Outcome Measures:
The secondary endpoints of the study are to assess the PK and IM. and IM of single fixed SC doses of a pharmaceutical dosage form of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one in adult subjects with moderately to severely active SLE. [Time Frame: Study Day 169]
PK:Individual and mean serum concentration-time profiles of a pharmaceutical dosage form of X by treatment group generated. IM: The presence of anti-drug antibodies against a pharmaceutical dosage form of X in serum is assessed and reported by number of subjects with detectable anti-drug antibodies and the percentage of positive subjects by treatment group. The titers of anti-drug antibodies in positive subjects will be reported.

Eligibility: Ages eligible for study: 18 years and older; Genders eligible for study: Both;

Inclusion Criteria:
Male or female subjects;
Age≦18 years at the time of screening;
Written informed consent and HIPAA authorization obtained from the subject/legal representative prior to performing any protocol-related procedures, including screening evaluations;
Meet or have met at least 4 of the 11 revised American College of Rheumatology (ACR) classification criteria for SLE (Appendix 2);
Score≧6 points on the Systemic Lupus Erythematosus Disease Activity Index 2000 (SLEDAI-2K) at screening and baseline;
Have positive antinuclear antibody (ANA) test at ≧1:80 serum dilution at screening;
Have active skin lesions from SLE in at least one area suitable for repeat skin biopsy, such as on the arms, legs, or trunk;
Females of childbearing potential, unless surgically sterile (ie, bilateral tubal ligation, bilateral oophorectomy, or complete hysterectomy), has sterile male partner, or post menopausal (defined as at least 2 years since last regular menses and follicle stimulating hormone (FSH)>23 IU/L according to central lab), or practices abstinence, must use 2 effective methods of avoiding pregnancy (including oral, transdermal, or implanted contraceptives, intrauterine device, female condom with spermicide, diaphragm with spermicide, cervical cap, or use of a condom with spermicide by the sexual partner) from screening, and must agree to continue using such precautions through the Early Discontinuation/End of Study (Day 169) visit; cessation of birth control after this point should be discussed with a responsible physician;
Males, unless surgically sterile, must use 2 effective methods of birth control with a female partner and must agree to continue using such contraceptive precautions from Day 1 through the Early Discontinuation/End of Study (Day 169) visit;
Ability to complete the study period, including follow-up period through Day 169;
Willingness to forego other forms of experimental treatment during the study.

Exclusion Criteria:
Pregnant or lactating woman;
History of alcohol or drug abuse≦1 year before screening, as judged by the investigator, before randomization into the study;
History of cancer except basal cell carcinoma treated with apparent success with curative therapy≧1 year before randomization into the study;
Elective surgery planned from the time of signing of the informed consent through end of study unless approved by the medical monitor;
Evidence of clinically significant lung pathology by chest x-ray (or chest computed tomography [CT]). A chest x-ray will be performed at screening, if one has not been performed in the last 6 months;
Evidence of active or latent tuberculosis (TB) unless adequately treated according to local guidelines for the treatment and prophylaxis of TB in immunocompromised patients;
History of primary immunodeficiency;
History of mixed connective tissue disease and overlap syndromes of SLE;
Evidence of infection at any time with hepatitis B or C virus or human immunodeficiency virus (HIV)-1 or HIV-2, or active infection with hepatitis A, as determined by results of testing at screening;
Any acute illness or evidence of clinically significant active infection between screening and Day 1;
History of sepsis or serious, recurrent, chronic infection, current signs and symptoms of clinically significant chronic infection, or recent (within 6 months before baseline visit) serious infection;
Deep space or tissue infections within 1 year of screening;
Any history or evidence of opportunistic infection within 6 months of screening including severe cytomegalovirus (CMV) or herpetic infections (such as disseminated herpes, herpes encephalitis, ophthalmic herpes);
History of any disease, evidence of any current disease (other than SLE) any finding upon physical examination, chest x-ray, or any laboratory abnormality that, in the opinion of the investigator or medical monitor, may compromise the safety of the subject in the study or confound the analysis of the study;
Any institutionalized individual;
Employees of the clinical study site or any other individuals involved with the conduct of the study, or immediate family members of such individuals;
History of allergic reactions to any component of the investigational product, diluent, or placebo;
Any oral or IV anti-infectives within 14 days before randomization into the study;
Vaccination with live attenuated viruses within 21 days before randomization into the study;
Receipt of the following concomitant medications within 21 days before randomization into the study:
   Mycophenolate mofetil>3 g/day;
   Systemic prednisone or equivalent>20 mg/day;
   Methotrexate and/or leflunomide;
   Topical treatments with immunosuppressants on the skin lesion to be biopsied (eg, corticosteroid creams, pimecrolimus);
Receipt of leflunomide>20 mg/day within 6 months before randomization into the study;
Receipt of cyclophosphamide (IV or oral) within 6 months of screening;
Change in daily doses or introduction of the following within 21 days before randomization into the study:
   Systemic corticosteroids;
   Antimalarials;
   Mycophenolate mofetil;
   Azathioprine;
Receipt of any investigational drug therapy within 21 days before randomization into the study, investigational T-cell-depleting therapies at any time, or other investigational biologic therapies within 30 days or 5 half-lives of administration of the biologic agent, whichever is longer, before randomization into the study;
Receipt of any biologic agents, within 30 days or 5 half-lives of administration of the biologic agent, whichever is longer, before randomization into the study;
Receipt of a B-cell depleting agent within 6 months of screening;
Have any absolute contraindications to skin punch biopsies, for example, a history of coagulation disorders;
At screening blood tests (within 21 days before randomization into the study), any of the following:
   Aspartate aminotransferase (AST)>1.5 upper limit of normal (ULN) unless caused by SLE, as determined by the investigator;
   Alanine aminotransferase (ALT)>1.5 ULN unless caused by SLE, as determined by the investigator;
   Creatinine>2.0 mg/dL;
   Neutrophils<1,000/mm3;
   Platelet count<75,000/mm3;
   Hemoglobin<7 g/dL;
   Hemoglobin Alc (HbAlc)>8% at screening (diabetic subjects only);
   Positive serum beta-human chorionic gonadotropin (β-hCG; pregnancy test);
An absolute CD4+ T cell count of <350 cells/mL within the 14 days before randomization into the study;
A urine protein:urine creatinine ratio of <3 by urine spot test at screening (within 21 days before randomization into the study);

### Example 11

### Pharmaceutical Compositions

The compositions described below are presented with (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one for illustrative purposes.

### Example 11a

### Parenteral Composition

To prepare a parenteral pharmaceutical composition suitable for administration by injection, 100 mg of a water-soluble salt of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is dissolved in DMSO and then mixed with 10 mL of 0.9% sterile saline. The mixture is incorporated into a dosage unit form suitable for administration by injection.

### Example 11b

### Oral Composition

To prepare a pharmaceutical composition for oral delivery, 100 mg of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is mixed with 750 mg of starch. The mixture is incorporated into an oral dosage unit for, such as a hard gelatin capsule, which is suitable for oral administration.

### Example 11c

### Sublingual (Hard Lozenge) Composition

To prepare a pharmaceutical composition for buccal delivery, such as a hard lozenge, mix 100 mg of (E)-14R)-3-(4-amino-3-(4-phenoxyphenyl)-1H-pyrazolo[3,4-d]pyrimidin-1-yl)pyrrolidin-1-yl)-4-(dimethylamino)but-2-en-1-one, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 11d

### Inhalation Composition

To prepare a pharmaceutical composition for inhalation delivery, 20 mg of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is mixed with 50 mg of anhydrous citric acid and 100 mL of 0.9% sodium chloride solution. The mixture is incorporated into an inhalation delivery unit, such as a nebulizer, which is suitable for inhalation administration.

### Example 11e

### Rectal Gel Composition

To prepare a pharmaceutical composition for rectal delivery, 100 mg of (E)-1-((R)-3-(4-amino-3-(4-phenoxyphenyl)-1H-pyrazolo[3,4-d]pyrimidin-1-yl)-4-pyrrolidin-1-yl)-4-(dimethylamino)but-2-en-1-one is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 11f

### Topical Gel Composition

To prepare a pharmaceutical topical gel composition, 100 mg of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 11g

### Ophthalmic Solution Composition

To prepare a pharmaceutical opthalmic solution composition, 100 mg of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is mixed with 0.9 g of NaCl in 100 mL of purified water and filterd using a 0.2 micron filter. The resulting isotonic solution is then incorporated into ophthalmic delivery units, such as eye drop containers, which are suitable for ophthalmic administration.

It is understood that the examples and embodiments described herein are for illustrative purposes only and that various modifications or changes in light thereof will be suggested to persons skilled in the art and are to be included within the spirit and purview of this application and scope of the appended claims

What is claimed is:

1. A pharmaceutical dosage form comprising from about 0.1 mg to about 40 mg of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or a pharmaceutically acceptable salt or solvate thereof.

2. The pharmaceutical dosage form of claim 1 comprising from about 0.1 mg to about 10 mg.

3. The pharmaceutical dosage form of claim 1 comprising from about 0.1 mg to about 5 mg.

4. The pharmaceutical dosage form of claim 1 comprising from about 0.5 mg to about 3.0 mg.

5. The pharmaceutical dosage form of any of claims 1 wherein the dosage form is suitable for once-a-day administration.

6. The pharmaceutical dosage form of any of claims 1 wherein the dosage form is suitable for oral administration.

7. The pharmaceutical dosage form of any of claims 1 further comprising a pharmaceutically acceptable carrier, excipient or binder.

8. A method for treating an immune-mediated disease or condition comprising administering to a subject in need thereof a therapeutically effective amount of from about 0.1 mg to about 40 mg of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one or a pharmaceutically acceptable salt or solvate thereof.

9. The method of claim 8 wherein the therapeutically effective amount is from about 0.1 mg to about 10 mg.

10. The method of claim 8 wherein the therapeutically effective amount is from about 0.1 mg to about 5 mg.

11. The method of claim 8 wherein the total amount of (E)-1-((R)-3-(4-amino-3-(4-phenoxyphenyl)-1H-pyrazolo[3,4-d]pyrimidin-1-yl)pyrrolidin-1-yl)-4-(dimethylamino)but-2-en-1-one is administered once a day.

12. The method of claim 8 wherein the immune-mediated disease or condition is inflammatory bowel conjunctivitis, allergic rhinitis, atopic dermatitis, or idiopathic thrombocytopenic purpura disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia, graft versus host disease, transplantation, transfusion, anaphylaxis, allergy, allergy-related urticaria, type I hypersensitivity, allergic.

13. The method of claim 8 wherein the immune-mediated disease or condition is rheumatoid arthritis, lupus, inflammatory bowel disease, allergy, allergy-related urticaria, multiple sclerosis, diabetes, idiopathic thrombocytopenic purpura or transplantation.

14. The method of claim 8 wherein the immune-mediated disease or condition is rheumatoid arthritis.
